(19) United States
(12) Patent Application Publication
(10) Pub. No.: US 2024/0410003 A1
(43) Pub. Date: Dec. 12, 2024

(54) microRNA BIOMARKERS

(71) Applicant: IMPERIAL COLLEGE INNOVATIONS LIMITED, London (GB)

(72) Inventors: Christopher Kyriacou, London (GB); Sung Hye Kim, London (GB); David Macintyre, London (GB); Phillip Bennett, London (GB); Tom Bourne, London (GB); Vasso Terzidou, London (GB)

(21) Appl. No.: 18/691,704

(22) PCT Filed: Sep. 16, 2022
(86) PCT No.: PCT/GB2022/052352

§ 371 Date: Mar. 13, 2024

(30) Foreign Application Priority Data
Sep. 17, 2021 (GB) 2113344.2

(51) Int. Cl. C12Q 1/6883

(52) U.S. Cl. CPC C12Q 1/6883; C12Q 2600/158; C12Q 2600/178

(57) ABSTRACT

The present invention provides a method for predicting an outcome of pregnancy, particular for predicting the likelihood of ectopic pregnancy (EP) or viable intrauterine pregnancy (VIUP), and methods for determining appropriate protocols for subjects with a pregnancy of unknown location (PUL) based on such an outcome, comprising determining the expression level of one or more specific miRNA molecules that are differentially expressed in scenarios of EP or VIUP, extracted from a biological sample obtained from said subject and comparing to a control value. The present invention also provides biochips and kits for use in carrying out the methods of the invention.

### CROSS-REFERENCE TO RELATED APPLICATIONS

This application is a 35 U.S.C. § 371 National Stage Entry of International Patent Application No. PCT/GB2022/052352, filed Sep. 16, 2022, which claims the benefit of priority of GB Application No. 2113344.2, filed Sep. 17, 2021, each of which is incorporated by reference herein in its entirety.

### SEQUENCE LISTING

The instant application contains a Sequence Listing which has been submitted electronically in XML file format and is hereby incorporated by reference in its entirety. Said XML Sequence Listing created on Feb. 20, 2024, is named “Sequence Listing ST26 240220 RMC00251US.xml” and is 3 KB in size.

### FIELD OF INVENTION

The present invention relates to microRNAs (miRNAs) that are useful in predicting pregnancy outcome, in particular the likelihood of ectopic pregnancy (EP), distinguishing between EP and viable intrauterine pregnancy (VIUP) or determining the likelihood of VIUP. The invention provides methods and kits for predicting the risk of any such complications in a subject with a pregnancy of unknown location (PUL), and for identifying pregnant subjects in need of interventions to treat non-viable pregnancies, such as EP.

### BACKGROUND

Pregnancy of unknown location (PUL) refers to a situation wherein the outcome of a pregnancy test is positive, yet a transvaginal ultrasound (TVUS) shows no signs of either an intrauterine or extrauterine pregnancy within a subject. Despite gradual improvements in high resolution ultrasonography equipment since the turn of the century, PUL remains a relatively common clinical scenario, with a varying prevalence across women attending early pregnancy clinics of between 5 and 42% (Boyraz, 2013).

Three critical outcomes of PUL have been recognised. A PUL can result in an intrauterine pregnancy (IUP) that can either be viable (VIUP), or non-viable NVIUP), or an ectopic pregnancy (EP). In a VIUP scenario, a visible foetal heartbeat of an embryo within a gestational sac can be identified within the uterus of the mother, suggesting the pregnancy has been established correctly. NVIUP refers to scenarios where, despite a pregnancy of a confirmed intrauterine location, the foetus has stopped growing and no heartbeat can be identified. In contrast to the above, an EP refers to abnormal extrauterine growth in which the embryo has attached outside the uterus, most commonly within the fallopian tube, where the embryo is unable to survive (Kirk et al., 2014).

Establishing the likely outcome of PUL is of crucial importance. An ectopic pregnancy occurs in 1-2% of pregnancies and is generally regarded as the most feared outcome of PUL (Boyraz, 2013). If diagnosed early, it is possible to medically treat EP with pharmaceutical intervention, such as methotrexate, or with surgical intervention. However, missing a diagnosis for EP can be highly detrimental to the mother; a possibility of rupture and intra-abdominal bleeding can bring significant risks of morbidity and mortality to the patient. Internal bleeding from such a rupture is the leading cause of death in the first trimester of the pregnancy (Christy, Kaczorowski & Garfunkel., 2007). One third of women with an ectopic pregnancy exhibit no medical signs and are therefore at high risk of complications, hence the critical need for an accurate early diagnosis (Kirk et al., 2014). Ascertaining the likely outcome of PUL would help to manage those patients at risk of an EP outcome and allow early intervention before any associated morbidities arise.

At present there exists no accurate predictor of EP or VIUP. Currently, the serum hormone human chorionic gonadotropin (hCG) is the most measured hormone in PUL patients, however its function as a predictor of outcome is imperfect. Instead of being used to predict EP or VIUP the hormone helps to determine whether its value falls above or below a so-called “discriminatory zone”, in which levels higher than the threshold indicate that an intrauterine gestational sac should be visible on an ultrasound (Pereira et al., 2019). In PUL cases where an hCG value is indeed above the discriminatory zone yet there is no visible intrauterine gestation, the result may be taken as a rough indication of an EP likelihood. However, this fails to consider the possibility of a viable IUP in which levels of hCG would also be higher than the threshold.

To further complicate matters, the values used to define the discriminatory zone often vary significantly across different practices without a unifying consensus. Serial hCG levels taken 48 hours apart are used to calculate what is known as the “hCG ratio”. Although better than discriminatory zones, using hCG ratios to define risk of ectopic pregnancy instead does not provide adequate accuracy. The approach is often combined with a test for foetal viability through measuring serum progesterone levels, yet this does little to resolve the location of a PUL. In general, recorded progesterone levels of below 5 ng/ml are associated with nonviable pregnancies, whilst levels of above 20 ng/ml indicate viable IUPs. Given that a considerable portion of EPs may present with variable progesterone readings of between 5 and 20 ng/mL, recording progesterone levels may also fail to rule out an EP (Pereira et al., 2019). More recently, progesterone and hCG have been combined within mathematical prediction models. These models are the current best way of predicting risk of PUL, however, methods with further precision are required (Christodoulou et al, 2020).

Without the ability to reach any certainty through current predictive techniques, there remains a difficulty to prepare for an outcome of VIUP or EP. Without ascertaining a clear difference between a likely VIUP and EP, to go forwards with medical or surgical intervention on a theorised EP has the potential to terminate an otherwise healthy embryo. Similarly, the imperfect predictive power of current techniques increases the risk of delayed or missed EP diagnoses and a lack of prevention of associated health conditions, putting greater pressure on health care systems, medical professionals and patients around the world.

Without any accurate biomarkers available for EP or VIUP there exists a need for novel markers that may provide certainty in predicting the outcome of a subject with PUL. Such predictive markers will help to standardise patient care, ensure all patients are safe, and prevent EP from being missed.

The listing or discussion of an apparently prior-published document in this specification should not necessarily be taken as an acknowledgment that the document is part of the state of the art or is common general knowledge.

### SUMMARY OF INVENTION

This invention is based on the surprising discovery that the expression level of certain microRNAs (miRNAs) in a biological sample correlate strongly with outcomes of EP and can therefore be used as a predictive biomarker for early detection of EP or VIUP in a pregnant subject. The expression level of particular miRNA molecules can therefore be used to determine a likely outcome of a PUL, helping to ensure the subject receives the correct therapeutic or surgical intervention, or follows the appropriate guidance for such an outcome. The present invention identifies particular miRNA biomarkers for the purpose described herein that are easily measurable and identifiable in the circulation of a subject/patient, allowing for a higher rate of patient compliance. Additionally, unlike current methods for predicting PUL outcome and diagnosis of ectopic pregnancy, the miRNA biomarkers herein described can be measured once, as a single visit protocol, providing the opportunity to reduce unnecessary resource expenditure and allowing more patients to be cared for safely and efficiently.

Accordingly, the miRNA biomarkers herein disclosed provide an improved method in which the prediction and diagnosis of ectopic pregnancy is made, preventing missed or delayed diagnoses and thus reducing surgical intervention, fertility reduction and significant morbidity or mortality.

In a first aspect, the invention provides a method for predicting the location and/or viability of an early pregnancy in a subject, the method comprising the step of determining the level of hsa-miR-411-5p in a biological sample obtained from the subject. Preferably, the subject has been classified as having a pregnancy of an unknown location prior to the biological sample being obtained.

In a second aspect, the invention provides a method for determining an appropriate medical management and/or treatment protocol for a pregnant subject, the method comprising the steps as defined herein.

In a third aspect, the invention provides a method of treating or managing a subject 5 with a pregnancy of unknown viability or location, the method comprising the steps of: (i) determining the level of hsa-miR-411-5p, hsa-miR-411-5p and hsa-miR-21-5p, or the ratio of expression of hsa-miR-21-5p to hsa-miR-411-5p in a biological sample obtained from the subject; and (ii) referring the subject for further medical examination and diagnosis and/or medical intervention.

In a fourth aspect, the invention provides a solid substrate comprising one or more probes that specifically hybridise to hsa-miR-411-5p and one or more probes that specifically hybridise to hsa-miR-21-5p, preferably wherein the solid substrate does not contain any further probes that specifically hybridise any other miRNA molecule.

In a fifth aspect, the invention provides a kit for predicting the viability and/or location of a pregnancy in a subject following conception, the kit comprising means to detect the level of hsa-miR-411-5p, the level of miR-411-5p and hsa-miR-21-5p, or the ratio of expression of hsa-miR-21-5p to hsa-miR-411-5p.

In a sixth aspect, the invention provides the use of an abortifacient in the manufacture of a medicament for the treatment of ectopic pregnancy (EP) in a patient who has been identified to have a decreased level of hsa-miR-411-5p, a decreased level of hsa-miR-411-5p and an elevated level of hsa-miR-21-5p or an increase in the ratio of expression of hsa-miR-21-5p to hsa-miR-411-5p in a biological sample obtained from that patient.

In a seventh aspect, the invention provides an abortifacient for use in the treatment of ectopic pregnancy in a patient, wherein the patient has been identified to have a decreased level of hsa-miR-411-5p, a decreased level of hsa-miR-411-5p and an elevated level of hsa-miR-21-5p or an increase in the ratio of expression of hsa-miR-21-5p to hsa-miR-411-5p in a biological sample obtained from said patient.

In an eighth aspect, the invention provides for a method of treating ectopic pregnancy in a patient who has been identified to have decreased level of hsa-miR-4115p, a decreased level of hsa-miR-411-5p and an elevated level of hsa-miR-21-5p or an increase in the ratio of expression of hsa-miR-21-5p to hsa-miR-411-5p, wherein the patient is subsequently administered an abortifacient, preferably wherein the abortifacient comprises a prostaglandin analogue and/or a progesterone receptor antagonist or antifolate.

### DESCRIPTION OF FIGURES

The invention is illustrated with reference to the following drawings, in which:

FIG. 1 shows the difference in fold change in hsa-miR-21-5p expression between subjects with VIUP (control group) and EP.

FIG. 2 shows the difference in fold change in hsa-miR-411-5p expression between subjects with VIUP (control group) and EP.

FIG. 3 shows the ROC curves for both hsa-miR-21-5p and hsa-miR-411-5p when used in isolation as biomarkers for predicting the location and/or viability of an early pregnancy in a subject classified with a pregnancy of unknown location.

FIG. 4 shows the difference in fold change between subjects with VIUP (control group) and EP when a ratio of hsa-miR-21-5p to hsa-miR-411-5p (21-5p/411-5p) is used for predicting the location and/or viability of an early pregnancy in a subject classified with a pregnancy of unknown location.

FIG. 5 shows the ROC curve for when the ratio of hsa-miR-21-5p to hsa-miR-411-5p (21-5p/411-5p) is used for predicting the location and/or viability of an early pregnancy in a subject classified with a pregnancy of unknown location.

### DETAILED DESCRIPTION OF THE INVENTION

This invention is predicated on the surprising discovery that the level of select miRNAs in biological samples obtained from pregnant female subjects, in comparison to control values, can be used to aid in predicting the likelihood of a pregnancy resulting in an ectopic pregnancy or viable intrauterine pregnancy. The miRNA molecules herein disclosed were found to be present in the maternal circulation and their concentration and relative levels correlated with outcomes of EP or VIUP. As such, profiling the level of the miRNAs herein disclosed may be used as an additional biomarker to increase the accuracy in predicting an outcome of a PUL, helping to ensure that an appropriate protocol is followed for the care of the subject.

Thus, in a first aspect, the invention provides a method for predicting the location and/or viability of an early pregnancy in a subject, the method comprising the step of determining the level of hsa-miR-411-5p in a biological sample obtained from the subject. Preferably, wherein the subject has been classified with a pregnancy of an unknown location.

The invention also provides a method for predicting the location of a pregnancy in a subject classified with a pregnancy of an unknown location, the method comprising the step of determining the level of hsa-miR-411-5p in a biological sample obtained from the subject. Preferably, wherein the method is for determining that the pregnancy is extrauterine. In particular, the method is for predicting the likelihood of the pregnancy being an ectopic pregnancy.

The term ectopic pregnancy (EP) refers to scenarios in which abnormal extrauterine growth occurs wherein the embryo has attached outside of the uterus, most commonly within the fallopian tube, in which the embryo is unable to survive. The term viable intrauterine pregnancy (VIUP) refers to a scenario wherein the foetal heartbeat of an embryo within a gestational sac can be identified within the uterus of the mother, suggesting the pregnancy has been established correctly.

The methods of the invention described herein are carried out ex vivo. For the avoidance of doubt, the term “ex vivo” has its usual meaning in the art, referring to methods that are carried out in or on a biological sample in an artificial environment outside the body of the patient from whom the biological sample has been obtained.

It is envisaged that the “biological sample” will be a blood sample but may be any other biological sample that is suitable for detecting miRNA molecules. For example, the sample may be a sample of amniotic fluid or urine. As used herein, the term “blood sample” includes whole blood and blood components, including plasma and serum. In preferred embodiments, the one or more miRNA molecules are extracted from the plasma component of a whole blood sample or from the serum component of a whole blood sample.

As used herein, “plasma” refers to the fluid portion of blood, excluding blood cells and platelets, but including dissolved proteins, glucose, clotting factors, electrolytes and hormones. As used herein, “serum” refers to blood plasma without clotting factors. The skilled person will be familiar with standard phlebotomy techniques which are suitable for obtaining a blood sample from a subject. The skilled person will also be familiar with routine techniques for obtaining plasma and/or serum from a whole blood sample, e.g. using centrifugation.

The terms “microRNA” and “miRNA” are used interchangeably herein and refer to small non-coding RNA molecules.

miRNAs are small, single-stranded, 19-25 nucleotide molecules that have emerged as important regulators of gene expression in almost all eukaryotes; a third of the protein encoding human genome is thought to be regulated by miRNAs. miRNAs are non-coding RNAs and function like small-interfering RNA to down-regulate gene expression at the post-transcriptional level. miRNA biogenesis involves a series of steps that lead to gene silencing. Briefly, miRNAs are transcribed in the nucleus as longer primary-miRNAs, which are cleaved to form hair-pin shaped precursor-miRNAs. These precursors are exported from the nucleus and further cleaved to form the mature miRNA which associates with the RNA induced silencing complex to target the 3′-untranslated region of specific mRNAs and inhibit their translation to protein. miRNAs are present in a cell free state in plasma and remain stable and easily measurable. Their potential utility as a biomarker of disease or response to treatment has consequently been widely acknowledged³.

miRNAs are expressed in a tissue specific manner and have differential expression, both spatially and over time. They remain stable and are easily detectable in blood and are thus suitable to provide novel, non-invasive biomarkers for outcomes of EP or VIUP according to the present invention. Advantageously, expression levels of these miRNA molecules can be measured from blood samples which can be obtained from female subjects in the early stages of pregnancy (from approximately gestation week 3 onwards, yet in some cases earlier still), thereby providing a minimally invasive means for the early prediction of an outcome of EP or VIUP.

The method herein described may comprise the step of determining the level of hsa-miR-411-5p and the level of hsa-miR-21-5p in a biological sample obtained from the subject. Both hsa-miR-411-5p and hsa-miR-21-5p when used in isolation may improve the predicting capability of currently used biomarkers in the context of predicting PUL. For example, hsa-miR-411-5p or hsa-miR-21-5p may be used in combination with hCG and/or progesterone to enhance the predictive value of using hCG and/or progesterone alone. However, it is a particularly surprising aspect of the invention that the specific combination of these two miRNAs, i.e. hsa-miR-411-5p and hsa-miR-21-5p, provide an even further enhanced predictive capability compared to using either one alone. Accordingly, the method of the present invention may further comprise the step of determining the level of hCG and/or progesterone in a or the sample obtained from the subject.

In a preferred embodiment, the method herein described comprises the step of determining the ratio of expression of hsa-miR-21-5p to hsa-miR-411-5p. As used herein, the term “ratio of expression” refers to the level of hsa-miR-21-5p expression divided by the level of hsa-miR-411-5p expression.

The invention herein disclosed provides a method wherein a decrease in the level of hsa-miR-411-5p in the biological sample may indicate an increased risk of ectopic pregnancy.

In a preferred embodiment, the invention provides a method wherein a decrease in the level of hsa-miR-411-5p in the biological sample and an increase in the level of hsa-miR-21-5p in the biological sample may indicate an increased risk of ectopic pregnancy (EP).

The terms “decrease” and “increase” in the context herein described, refer to a decrease or increase in the levels of hsa-miR-411-5p and hsa-miR-21-5p relative to the level of said miRNA's in a control sample, for example, a biological sample obtained from a subject with a PUL classification who has then been determined to have a viable intrauterine pregnancy.

In a most preferred embodiment, the invention provides a method wherein an increase in the ratio of expression of hsa-miR-21-5p to hsa-miR-411-5p in the biological sample, compared to the levels obtained from a control subject, may indicate an increased risk of ectopic pregnancy (EP).

A lack of significant change in the level of hsa-miR-411-5p, in the level of hsa-miR-411-5p and hsa-miR-21-5p, or in the ratio of expression of hsa-miR-21-5p to hsa-miR-411-5p, compared to the levels obtained from a control subject, may indicate that there is no increased risk of EP or that the pregnancy may result in a viable intrauterine pregnancy (VIUP).

By “predicting” we include the meaning of aiding in diagnosing. Thus, if the one or more miRNA molecule that is detected using the method according to this aspect of the invention is a molecule whose over-expression is associated with increased risk of EP, then an elevated expression level of said miRNA in the subject's sample compared to the control value is indicative of a likely outcome of EP in said subject, and said subject may be diagnosed with EP.

Similarly, if the one or more miRNA molecule that is detected using any method according to the invention is a molecule whose level is not over-expressed above levels associated VIUP, then an expression level of said miRNA in the subject's sample compared to the control value is indicative of a likely outcome of VIUP in said subject.

As used herein, the term “control value” refers to a baseline level of the corresponding one or more miRNA molecules in a corresponding control sample. The corresponding control sample may be obtained from a cohort of pregnant female subjects at an appropriate stage of pregnancy who had an outcome of VIUP. The selection of appropriate control samples and parameters is well within the capability of the skilled person.

In some embodiments, the control value is obtained from the same subject before pregnancy. In other embodiments, the control value may be obtained from a control subject who is at the same stage of pregnancy to the subject from whom the biological sample is being obtained. In other embodiments, the control value may be obtained from a subject classified with a PUL who has then been determined to have a viable intrauterine pregnancy.

In a preferred embodiment, the control value is determined in a biological sample obtained from a pregnant subject already determined to have had viable intrauterine pregnancy, optionally wherein the subject is determined to have had viable pregnancy through means such as ultrasound screening or as a result of a healthy birth.

If the level of two or more miRNA molecules is determined in the methods of the invention, for example, hsa-miR-411-5p and hsa-miR-21-5p, the corresponding control value is the combined baseline level of the corresponding miRNAs in a control sample, for example, a sample obtained from a subject classified with a PUL who has then been determined to have a viable intrauterine pregnancy.

Preferably, for each of the methods of the invention, the cut-off value for determining whether the level of a given miRNA molecule is “different” (elevated or reduced) compared with a control value is 2-times the baseline level for the miRNA. Therefore, if the level of a given miRNA (e.g. hsa-miR-411-5p) is determined and the value is found to be at least 2-fold lower than the baseline level for hsa-miR-411-5pin a control sample, then it can be concluded that hsa-miR-411-5p is decreased in the subject's sample and a prediction of outcome can be made, in accordance with the methods of the invention. Likewise, if the level of a given miRNA (e.g. hsa-miR-21-5p) is determined and value is found to be at least 2-fold greater than the baseline level of hsa-miR-21-5p in a control sample, then it can be concluded that hsa-miR-21-5p is elevated in the subject's sample and a prediction of outcome can be made, in accordance with the methods of the invention.

The term “level” is synonymous with “expression level” and is used broadly to include a genomic expression profile, e.g. an expression profile of miRNAs. The level of the one or more miRNA molecules in the patient's sample and/or the control sample can be determined using any convenient means for determining a level of a nucleic acid sequence, e.g. quantitative nucleic acid hybridisation of miRNA, labelled miRNA, and/or nucleic acid amplification techniques which are routinely used in the art and which the skilled person will be familiar with.

Preferred techniques for determining the miRNA level include: real-time PCR (RT-PCR), a technique suitable for large scale/multiple analysis and useful for screening large populations; microarray, comprising a 2D array on a solid substrate; next generation sequencing platforms for example RNAseq, in which the advantages of next generation sequencing include its high throughput, speed and low cost per base; and in situ hybridisation.

The present inventors have identified the combined analysis of the level of hsa-miR-411-5p and hsa-miR-21-5p as being particularly useful in the context of the present invention. For the avoidance of doubt, “hsa-” refers to Homo sapiens and is a standard abbreviation to differentiate the miRNAs from those of other species. The suffixes “3p” and “5p” denote 3 prime or 5 prime, respectively. These suffixes are used to distinguish two miRNAs originating from opposite arms of the same pre-miRNA.

All miRNAs are identified herein using standard nomenclature. Sequence information for each of the miRNAs listed in Table 1 can be found on the miRBase database maintained by Manchester University (http://mirbase.org/search.shtml).

It will be apparent to the person skilled that the methods of the invention disclosed herein can be used in conjunction with other methods for screening for EP and predicting viability of a pregnancy that are well known in the art.

The terms “patient” and “subject” are used interchangeably herein and refer to any female animal (e.g. mammal), including, but not limited to, humans, non-human primates, canines, felines, rodents and the like. Thus, in some embodiments the subject is any female animal. In a preferred embodiment, the subject is a human female.

The biological sample according to any method of the invention can be taken from the mother at any stage during pregnancy. In some embodiments the sample may be taken during the first trimester. In a preferred embodiment the sample may be taken at any stage until 10 weeks gestation. In a more preferred embodiment, the sample may be taken between at any stage until 9 weeks gestation. In a most preferred embodiment, the sample may be taken between 4 and 9 weeks gestation, or between 0 and 4 weeks gestation.

If, as a result of carrying out the method of the invention, the patient is identified as having a likely outcome of EP, then further diagnostic testing or monitoring can be carried out and/or the patient can be treated with therapeutic or surgical interventions, which aim to prevent the continuing growth of an ectopic pregnancy.

Such interventions would be known within the art and may include the administering of an abortifacient. In some embodiments the abortifacient may comprise a prostaglandin analogue and/or a progesterone receptor antagonist or antifolate. In a preferred embodiment the prostaglandin analogue may be misorprostol or gemepost. In a further preferred embodiment the progesterone receptor antagonist may be mifepristone. In yet another further preferred embodiment the antifolate may be methotrexate (MXT), administered by injection to stop cell growth and breakdown existing cells.

In other embodiments it is envisaged that surgical interventions may take place, in which the subject is treated with laparoscopic surgery.

Laparoscopic surgery refers to a procedure wherein a small incision is made in the abdomen, near or in the navel of the subject. A thin tube equipped with a camera lens and light, referred to as a laparoscope, is used to view the tubal area, before the ectopic pregnancy is removed, and the tube is either repaired (salpingostomy) or removed (salpingectomy).

It would also be appreciated by the skilled person that if, as a result of carrying out the method of the invention, the subject is identified as having a likely outcome of VIUP, then alongside further diagnostic testing or monitoring, the patient may be encouraged to follow normal procedural guidance for an uncomplicated pregnancy.

It is envisaged that such normal procedural guidance may refer to official national or local guidelines for normal pregnancy established across a particular area or within a particular hospital or clinic. Such guidelines may comprise attending appropriate prenatal care and undergoing suitable medical check-ups and screening tests. Such check-ups may typically occur once each month between 4 and 28 weeks gestation, twice a month for weeks 28 to 36, and weekly for weeks 36 to birth.

It is also envisaged that the subject may be instructed to follow certain lifestyle and dietary protocols, such as limiting smoking and alcohol consumption, and increasing folic acid intake.

Thus, in some embodiments of the present invention wherein the subject is identified as having a likely outcome of VIUP, the subject may be instructed to follow procedural guidance for an uncomplicated pregnancy. In a preferred embodiment, the subject may be instructed to follow procedural guidance for an uncomplicated pregnancy, wherein said guidance comprises attending suitable medical check-ups and screening tests, and/or following certain lifestyle and dietary protocols, such as limiting smoking and alcohol consumption, and increasing folic acid intake.

The term pregnancy of unknown location (PUL) refers to a situation wherein the outcome of a pregnancy test is positive, yet a transvaginal ultrasound (TVUS) shows no signs of either an intrauterine or extrauterine pregnancy within a subject. In some embodiments the method for predicting EP or VIUP according to the present invention may be carried out on any pregnant female subject. In a preferred embodiment, the method may be carried out on a subject with a PUL classification.

In a second aspect, the invention provides a method for determining an appropriate medical management and/or treatment protocol for a pregnant subject, the method comprising the steps as defined herein. Accordingly, the invention provides a method for determining an appropriate medical management and/or treatment protocol for a pregnant subject, wherein the method comprises the step of determining the level of hsa-miR-411-5p, determining the level of hsa-miR-411-5p and hsa-miR-21-5p or determining the ratio of expression of hsa-miR-21-5p to hsa-miR-411-5p.

Specifically, the invention provides a method for determining an appropriate medical management and/or treatment protocol for a pregnant subject, wherein the level of hsa-miR-411-5p is decreased compared to a control value, wherein the level of hsa-miR-411-5p is decreased at the same time as the level of hsa-miR-21-5p is increased compared to a control value, or wherein the ratio of expression of hsa-miR-21-5p to hsa-miR-411-5p is increased compared to a control value indicates an increased risk of EP. Accordingly, the subject may be referred for further medical examination and diagnosis and/or medical intervention. Preferably, said medical intervention comprises the administration of an abortifacient and/or surgery.

A lack of significant change of the level the miRNAs herein disclosed, for example, hsa-miR-411-5p and hsa-miR-21-5p may indicate that there is no increased risk of EP or that the pregnancy may result in a VIUP. Accordingly, in these instances, the subject may be instructed to follow procedural guidance for an uncomplicated pregnancy and have no requirement for further medical management and/or treatment in the context of EP.

In a third aspect, the invention provides a method of treating or managing a subject with a pregnancy of unknown viability or location, the method comprising the steps of: (i) determining the level of hsa-miR-411-5p, hsa-miR-411-5p and hsa-miR-21-5p, or the ratio of expression of hsa-miR-21-5p to hsa-miR-411-5p in a biological sample obtained from the subject; and (ii) referring the subject for further medical examination and diagnosis and/or medical intervention.

In a fourth aspect, the invention provides a solid substrate comprising one or more probes that specifically hybridise to hsa-miR-411-5p and one or more probes that specifically hybridise to hsa-miR-21-5p, preferably wherein the solid substrate does not contain any further probes that specifically hybridise any other miRNA molecule. The solid substrate, which may also be referred to as a support material, is preferably a biochip. The probes may be attached thereto or immobilised thereon the solid substrate.

As used herein, the term “probe” refers to any binding molecule capable of binding to a target nucleic acid (i.e. a miRNA molecule) of complimentary sequence. Accordingly, probes may refer to oligonucleotides, aptamers and/or antibodies. Probes may bind to targets lacking complete complementarity with the probe sequence, depending upon the stringency of the hybridisation conditions. Probes may be directly labelled or indirectly labelled, such as with biotin to which a streptavidin complex may later bind.

The probes may be capable of hybridising to a target miRNA sequence under stringent hybridisation conditions. The probes may be attached at spatially defined locations on the substrate. The solid substrate may be a material that may be modified to contain discrete individual sites appropriate for the attachment or association of the probes and is amenable to at least one detection method. Examples of suitable substrates include glass and modified or functionalised glass, plastics, polysaccharides, nylon or nitrocellulose, resins, silica or silica-based materials, carbon and metals. The substrate may allow optical detection without appreciably fluorescing.

The substrate may be planar, although other configurations of substrates may be used as well. For example, probes may be positioned on the inside surface of a tube.

The solid substrate and the probe may be derivatised with a chemical functional group, such that the probe may be attached using the functional group directly or indirectly using a linker. Alternatively, the probe may be attached to the solid support non-covalently, for example using biotinylated oligonucleotides. Alternatively, the probe may be synthesised on the surface of the solid support using techniques such as photopolymerisation and photolithography.

Preferably, the solid substrate comprises binding molecules specific to each of hsa-miR-411-5p and hsa-miR-21-5p. As used herein, the term “binding molecules” may refer to any molecule capable of specifically binding to hsa-miR-411-5p or hsa-miR-21-5p, for example, a binding molecule may refer to, but is not limited to, an antibody, an aptamer or an oligonucleotide sequence. Preferably, the solid substrate comprises oligonucleotide sequences. The support material can be used in a method according to any of the first, second or third aspects of the invention.

The invention also provides the use of a solid substrate in a method according to the invention.

In a fifth aspect, the invention provides a kit for predicting the viability and/or location of a pregnancy in a subject following conception, the kit comprising means to detect the level of hsa-miR-411-5p, the level of miR-411-5p and hsa-miR-21-5p, or the ratio of expression of hsa-miR-21-5p to hsa-miR-411-5p.

Preferably the kit comprises means to detect only the above listed miRNAs. The means may be a probe as set out herein.

The kit may comprise any or all of the following: assay reagents, buffers, probes and/or primers, sterile saline or another pharmaceutically-acceptable emulsion and suspension base. In addition still, the kits may include instructions for use for the practice of the methods described herein.

In some embodiments, the kit may be used to carry out any of the methods described herein.

In a sixth aspect, the invention provides the use of an abortifacient in the manufacture of a medicament for the treatment of ectopic pregnancy (EP) in a patient, wherein the patient has been identified to have decreased level of hsa-miR-4115p, a decreased level of hsa-miR-411-5p and an elevated level of hsa-miR-21-5p or an increase in the ratio of expression of hsa-miR-21-5p to hsa-miR-411-5p in a biological sample obtained from that patient.

In a seventh aspect, the invention provides an abortifacient for use in the treatment of ectopic pregnancy, wherein the patient has been identified to have a decreased level of hsa-miR-411-5p, a decreased level of hsa-miR-411-5p and an elevated level of hsa-miR-21-5p or an increase in the ratio of expression of hsa-miR-21-5p to hsa-miR-411-5p in a biological sample obtained from said patient.

In a preferred embodiment, the abortifacient comprises a prostaglandin analogue and/or a progesterone receptor antagonist or antifolate.

In an eighth aspect, the invention provides for a method of treating a patient with ectopic pregnancy, wherein the patient has been identified to have decreased level of hsa-miR-411-5p, a decreased level of hsa-miR-411-5p and an elevated level of hsa-miR-21-5p or an increase in the ratio of expression of hsa-miR-21-5p to hsa-miR-411-5p, wherein the patient is subsequently administered an abortifacient, preferably wherein the abortifacient comprises a prostaglandin analogue and/or a progesterone receptor antagonist or antifolate.

### Sequences

The following table comprises the sequences listed within the present application and each corresponding SEQ ID NO:

TABLE 1

| miRNA | SEQ ID NO | Sequence |
|---|---|---|
| hsa-miR-21-5p | SEQ ID | UAGCUUAUCAGACUGAUGUUGA |
| | NO: 1 | |
| hsa-miR-411-5p | SEQ ID | UAGUAGACCGUAUAGCGUACG |
| | NO: 2 | |

The invention will be further described with reference to the following non-limiting examples.

The contents of any document mentioned herein are hereby incorporated by reference in its entirety.

### EXAMPLES

The inventors of the present invention have surprisingly found that determining the level of expression of particular miRNAs, for example, hsa-miR-411-5p and hsa-miR-21-5p, correlates strongly with outcomes of EP and can therefore be used as predictive markers for early detection of EP or VIUP in a pregnant subject.

The inventors have further discovered that determining the level of hsa-miR-411-5p and hsa-miR-21-5p in combination, opposed to in isolation, and specifically as a ratio of one to another, results in an even more enhanced predictive capability.

Hsa-miR-411-5p and Hsa-miR-21-5p have Altered Expression in EP Subjects Compared to Control Subjects

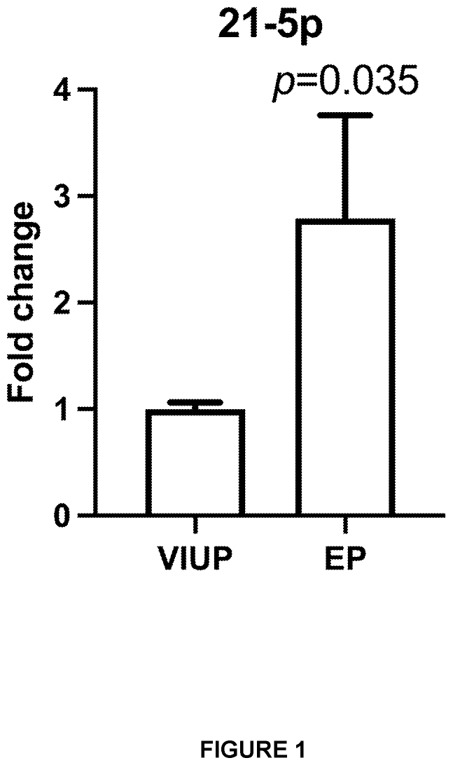

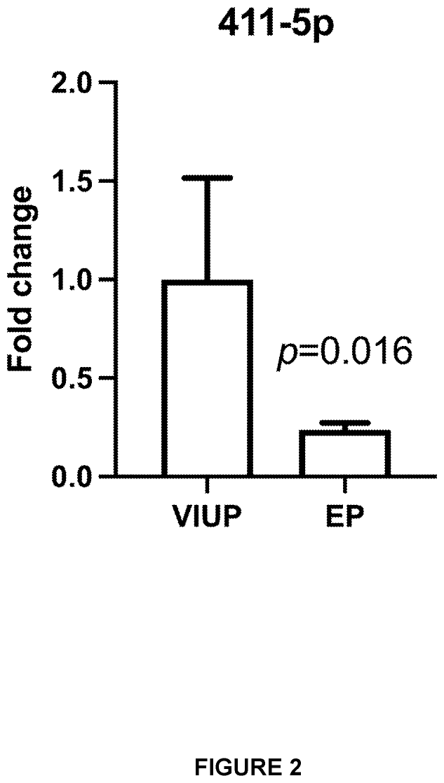

The inventors of the present invention have demonstrated that both miRNAs have altered levels of expression when measured in EP subjects compared to control subjects, i.e. subjects with VIUP. Specifically, hsa-miR-21-5p is found to be increased in EP subjects when compared with control subjects (see FIGS. 1 and Table 2) and hsa-miR-411-5p is found to be decreased in EP subjects when compared with control subjects (see FIG. 2 and Table 2). The difference in expression for each miRNA molecule is displayed as a fold change over the control value i.e. 1, calculated from RT-qPCR data. The increase in hsa-miR-21-5p expression was found to be 2.79 fold in the EP subjects when compared with the control subjects. The decrease in hsa-miR-411-5p expression was found to be 0.24 fold in the EP subjects when compared with the control subjects. Both the increase in hsa-miR-21-5p expression and the decrease in hsa-miR-411-5p expression were found to be significant changes from the control group, with p values of 0.035 and 0.016, respectively.

TABLE 2
Fold change of hsa-miR-21-5p and hsa-miR-411-5p expression

| Fold Change | hsa-miR-21-5p | hsa-miR-411-5p |
|---|---|---|
| VIUP | 1 | 1 |
| EP | 2.791842 | 0.236949 |

The accuracy of a diagnostic method is often described by its receiver-operating characteristics (ROC), from which an ROC curve can be generated. The ROC curve is a plot of all the sensitivity/specificity pairs resulting from continuously varying the decision threshold over the entire range of data observed. A ROC plot depicts the overlap between the two distributions by plotting the sensitivity versus 1-specificity for the complete range of decision thresholds. On the y-axis is sensitivity, or the positive fraction defined as [(number of true-positive test/(number of true-positive+number of false-negative test results)]. This has also been referred to as positivity in the presence of a disease or condition. It is calculated solely from the affected subgroup. On the x-axis is the false-positive fraction, or 1-specificity defined as [(number of false-positive results)/(number of true-negative+number of false-positive results)]. It is an index of specificity and is calculated entirely from the unaffected subgroup. As the true- and false-positive fractions are calculated entirely separately, by using the test results from two different subgroups, the ROC plot is independent of the prevalence of disease in each sample. Each point on the ROC plot represents a sensitivity/specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination is a 45 degree diagonal line from the lower left corner to the upper right corner. Most plots fall in-between these two extremes.

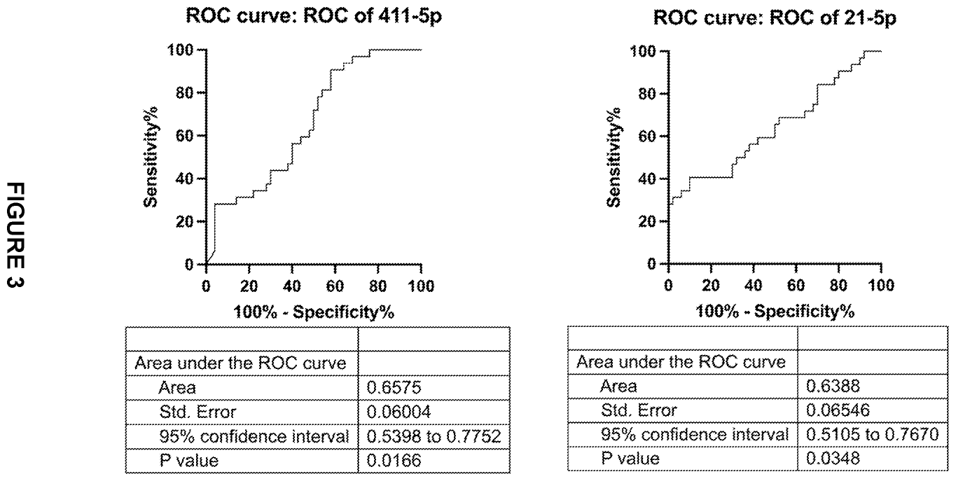

Qualitatively, the closer the plot is to the upper left corner, the higher the overall accuracy of the test. A convenient goal to quantify the diagnostic accuracy is to use the area under the curve (AUC) of the ROC plot, with a value of 1 signifying a perfect ROC plot. Accordingly, the closer the AUC value is to 1 the better the diagnostic accuracy of the test being assessed. The ROC curves for using hsa-miR-21-5p and hsa-miR-411-5p as predictive markers are shown in FIG. 3. The AUC values are 0.639 and 0.658 respectively, demonstrating a good level of accuracy.

Hsa-miR-411-5p and Hsa-miR-21-5p when Analysed in Combination have Improved Predictive Capability Compared to when Analysed in Isolation

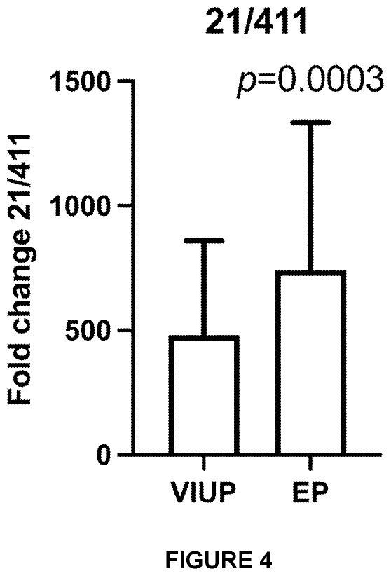

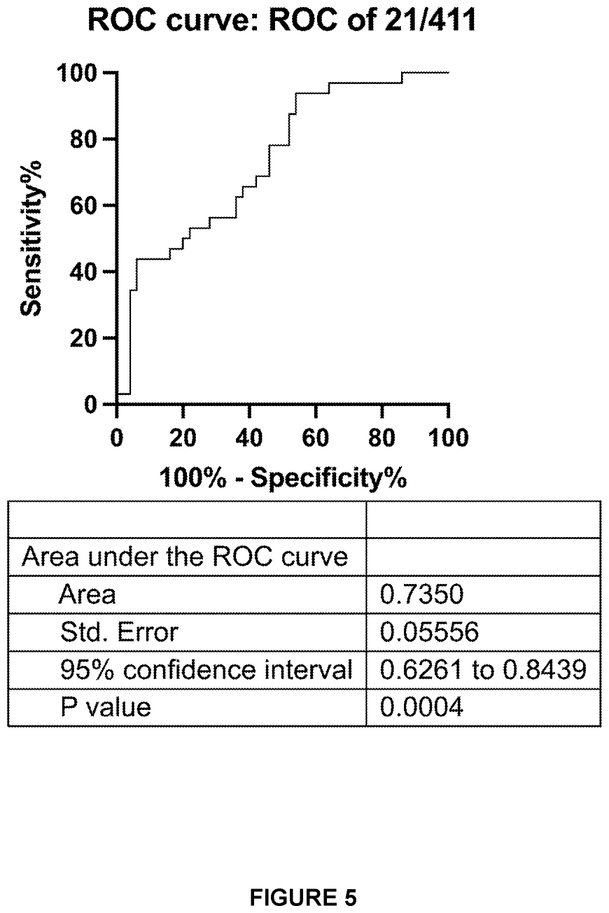

It is a surprising finding of the present invention that hsa-miR-411-5p and hsa-miR-21-5p when used in combination, and in particular as a ratio, provide for an improved predictive capability of subjects with EP when compared to using either one alone. This is demonstrated in FIGS. 4 and 5, wherein the ratio of these miRNA biomarkers, i.e. hsa-miR-21-5p/hsa-miR-411-5p, is shown to be significantly increased in EP subjects compared to VIUP subjects and the AUC value is 0.735, a marked improvement from the AUC values of using either one alone.

Accordingly, the data herein demonstrates the suitability of using the miRNA markers herein disclosed in a method for predicting the location and/or viability of an early pregnancy in a subject classified with a pregnancy of unknown location.

1. A method for predicting the location and/or viability of an early pregnancy in a subject, the method comprising the step of determining the level of hsa-miR-411-5p in a biological sample obtained from the subject.

2. The method of claim 1, wherein the method comprises the step of determining the level of hsa-miR-411-5p and the level of hsa-miR-21-5p in a biological sample obtained from the subject.

3. The method of claim 2, wherein the method comprises the step of determining the ratio of expression between hsa-miR-411-5p and hsa-miR-21-5p.

4. The method of claim 1, wherein the method is for predicting the likelihood of ectopic pregnancy (EP) in the subject.

5. The method of claim 1, wherein a decrease in the level of hsa-miR-411-5p in the biological sample indicates an increased risk of ectopic pregnancy (EP).

6. The method of claim 2, wherein an increase in the level of hsa-miR-21-5p in the biological sample indicates an increased risk of ectopic pregnancy (EP).

7. The method of claim 3, wherein an increase in the ratio of expression of hsa-miR-21-5p to hsa-miR-411-5p in the biological sample indicates an increased risk of ectopic pregnancy (EP).

8. The method of claim 1, wherein no significant change in the level of hsa-miR-411-5p, in the level of hsa-miR-411-5p and hsa-miR-21-5p, or in the ratio of expression of hsa-miR-21-5p to hsa-miR-411-5p indicates that there is no increased risk of EP or that the pregnancy may result in a viable intrauterine pregnancy (VIUP).

9. The method of claim 1, wherein the level of the miRNA is compared with a control level to determine if there is a change in the level; preferably wherein the control level is determined in a biological sample obtained from the same subject before pregnancy, or from a biological sample obtained from a pregnant subject already determined, or later determined, to have had a viable intrauterine pregnancy; optionally wherein the subject is determined to have had viable pregnancy through means such as ultrasound screening or as a result of a healthy birth.

10. The method of claim 5, wherein following the determination of an increased risk of ectopic pregnancy, the subject is referred for further medical examination and diagnosis, and/or referred for medical intervention; optionally wherein the medical intervention comprises administration of an abortifacient and/or surgery; optionally wherein the abortifacient comprises a prostaglandin analogue and/or a progesterone receptor antagonist or antifolate; preferably wherein the prostaglandin analogue is misoprostol or gemeprost and/or wherein the progesterone receptor antagonist is mifepristone and/or the antifolate is methotrexate (MTX).

11. The method of claim 8, wherein the subject is instructed to follow procedural guidance for an uncomplicated pregnancy.

12. The method of claim 1, wherein the method further comprises the step of determining the level of hCG and/or progesterone in a sample obtained from the subject.

13. A method for determining an appropriate medical management and/or treatment protocol for a pregnant subject, the method comprising the steps as defined in claim 1.

14. A method of treating or managing a subject with a pregnancy of unknown viability or location, the method comprising the steps of:
(i) determining the level of hsa-miR-411-5p, hsa-miR-411-5p and hsa-miR-21-5p, or the ratio of expression of hsa-miR-21-5p to hsa-miR-411-5p in a biological sample obtained from the subject; and
(ii) referring the subject for further medical examination and diagnosis and/or medical intervention wherein a decrease in the level of hsa-miR-411-5p is determined, an increase in the level of hsa-miR-21-5p is determined, or an increase in the ratio of expression of hsa-miR-21-5p to hsa-miR-411-5p is determined.

15. The method of claim 14, wherein the medical intervention comprises administration of an abortifacient and/or surgery.

16. The method of claim 14, the method comprising the steps as defined in claim 8.

17. The method of claim 14, wherein the biological sample is a blood or plasma sample.

18. The method of claim 14, wherein the method is carried out on samples obtained from the subject when the subject is between 4 to 9 weeks gestation.

19. The method of claim 14, wherein the level of the miRNA molecules is determined using nucleic acid hybridisation and/or nucleic acid amplification; preferably wherein the level of the miRNA molecules is determined using real-time quantitative PCR, microarray, Next Generation Sequencing platforms, or in situ hybridisation; more preferably wherein the level of the miRNA molecules are determined using real-time quantitative PCR.

20. (canceled)

21. (canceled)

22. A kit for predicting the viability and/or location of a pregnancy in a subject following conception, the kit comprising means to detect the level of hsa-miR-411-5p, the level of miR-411-5p and hsa-miR-21-5p, or the ratio of expression of hsa-miR-21-5p to hsa-miR-411-5p; preferably wherein the means to detect the level of the miRNA molecules are oligonucleotides, optionally wherein at least a part of the sequence of the oligonucleotide is complementary to at least part of the miRNA nucleic acid sequence.

23. The kit of claim 22, wherein the means to detect the level of the miRNA molecules are immobilised to a solid surface.

24. The kit of claim 22, further comprising one or more of assay reagents, buffers, probes and/or primers, a pharmaceutically-acceptable emulsion and suspension base, and instructions for use.

25. (canceled)


SEQUENCE LISTING (2 sequences)

SEQ ID NO: 1 - RNA, 22 nt, Homo sapiens
  source 1..22: mol_type = other RNA
tagcttatcagactgatgttga

SEQ ID NO: 2 - RNA, 21 nt, Homo sapiens
  source 1..21: mol_type = other RNA
tagtagaccgtatagcgtacg